(19) United States
(12) Patent Application Publication
(10) Pub. No.: US 2019/0153048 A1
(43) Pub. Date: May 23, 2019

(54) SYSTEM AND METHOD FOR THE PRODUCTION OF A BIOLOGICAL MESH

(71) Applicant: The Medical College Of Wisconsin, Inc., Milwaukee, WI (US)

(72) Inventor: Kenneth Matthew Scaglione, Milwaukee, WI (US)

(21) Appl. No.: 16/102,952

(22) Filed: Aug. 14, 2018

Related U.S. Application Data
(60) Provisional application No. 62/546,712, filed on Aug. 17, 2017.

(51) Int. Cl. C07K 14/44; C07K 14/415; C08F 20/06; A61K 9/70

(52) U.S. Cl. CPC C07K 14/44; A61K 9/70; C08F 20/06; C07K 14/415

(57) ABSTRACT

The present disclosure provides a system and method for generating a biological mesh. In one embodiment, the biological mesh comprises one or more heat shock protein domains, where the one or more heat shock protein domains comprise at least a portion of a C-terminus of a Dictyostelium discoideum Hsp48 protein. The biological mesh further comprises a polymeric material having a negative charge, the polymeric material comprising polyphosphate having a number of monomer units [PO₃⁻]ₙ, wherein n is greater than 100.

### CROSS-REFERENCE TO RELATED APPLICATIONS

This application claims priority to U.S. Provisional application 62/546,712, filed Aug. 17, 2017 and incorporated by reference herein.

### STATEMENT OF FEDERALLY SPONSORED RESEARCH OR DEVELOPMENT

This invention was made with government support under 5R00NS073936 awarded by the National Institutes of Health. The government has certain rights in the invention.

### BACKGROUND

Upon cellular stress, cells elicit a number of responses, including induction of stress responsive genes, reduced translation, formation of phase-separated compartments including stress granules, and production of the chemical chaperone polyphosphate. Activation of these pathways provides defense mechanisms for cells to combat protein aggregation and promote cell survival. In addition to activating stress pathways, some organisms enter dormancy, allowing them an opportunity to survive adverse conditions, including extreme temperatures, desiccation, and starvation (1).

In response to unfavorable conditions, the social amoeba Dictyostelium discoideum (herein after “Dictyostelium”) undergoes a developmental process where it transitions from a single cellular organism, culminating in the formation of a multicellular fruiting body that contains dormant spores. Dictyostelium spores can survive unfavorable conditions like extreme temperatures and desiccation (1). The spores consist of a thick cellulose coat that encapsulates a dehydrated amoeba which germinates upon alleviation of a stressor. Dictyostelium encodes a unique proteome that contains a large number of repetitive amino acid tracts that remains resistant to protein aggregation through an undetermined mechanism (2, 3). While little is known about protein quality control pathways during Dictyostelium spore development, the primordial chemical chaperone polyphosphate dramatically increases during this period (4, 5), suggesting that polyphosphate may prevent protein aggregation in Dictyostelium spores.

In response to unfavorable conditions, the social amoeba Dictyostelium discoideum (herein after “Dictyostelium”) undergoes a developmental process where it transitions from a single cellular organism, culminating in the formation of a multicellular fruiting body that contains dormant spores. Dictyostelium spores can survive unfavorable conditions like extreme temperatures and desiccation (1). The spores consist of a thick cellulose coat that encapsulates a dehydrated amoeba which germinates upon alleviation of a stressor. Dictyostelium encodes a unique proteome that contains a large number of repetitive amino acid tracts that remains resistant to protein aggregation through an undetermined mechanism (2, 3). While little is known about protein quality control pathways during Dictyostelium spore development, the primordial chemical chaperone polyphosphate dramatically increases during this period (4, 5), suggesting that polyphosphate may prevent protein aggregation in Dictyostelium spores.

Currently, there is a need in the art to create a biological mesh comprising a Dictyostelium discoideum Hsp48.

### SUMMARY OF THE PRESENT DISCLOSURE

The present disclosure relates to a phase-separated biological mesh composition comprising at least a portion of a previously uncharacterized alpha-crystallin-domain-containing protein, a Dictyostelium discoideum Hsp48, and a polymeric material having a negative charge. In various embodiments of the invention, the biological mesh composition may be coupled to therapeutics or may be configured to entrain therapeutics for use in drug, gene, and vaccine delivery. It is further contemplated that the biological mesh may be used to form improved biomaterials and bioelectronics.

Some aspects of the present disclosure provide a biological mesh. The biological mesh comprises one or more heat shock protein domains, where the one or more heat shock protein domains comprise at least a portion of a C-terminus of a Dictyostelium discoideum Hsp48 protein.

The biological mesh further comprises a polymeric material having a negative charge.

In one embodiment, the polymeric material comprises polyphosphate having a number of monomer units [PO₃⁻]ₙ, wherein n is greater than 100.

Other aspects of the present disclosure provide a biological mesh comprising one or more heat shock protein domains, wherein the one or more heat shock protein domains comprise at least a portion of a C-terminus of a Dictyostelium discoideum Hsp48 protein. The biological mesh further comprising a polymeric material comprising a polyelectrolyte or an ionomer having a repeating unit coupled to one or more electrolyte group, wherein the electrolyte group is selected from the group consisting of —COOH, —SO₃H, and —PO₃H₂.

Some aspects of the present disclosure provide a biological mesh comprising one or more heat shock protein domains, where the one or more heat shock protein domains comprise at least a portion of a C-terminus of a Dictyostelium discoideum Hsp48 protein. The biological mesh further comprises a polymeric material having a negative charge, the polymeric material having a western blot densitometry that is greater than a reference western blot densitometry, where the reference western blot densitometry includes a phase separated sample taken from a Dictyostelium discoideum amoeba.

Other aspects of the present disclosure provide a method for producing a biological mesh. The steps of the method comprise providing a solution where the solution has a liquid phase and adding at least a portion of a C-terminus domain of a Dictyostelium discoideum Hsp48 protein and a polymeric material having a negative charge to the solution. The solution undergoes a phase separation to generate the biological mesh.

These and still other advantages of the invention will be apparent from the detailed description and drawings. What follows is merely a description of some preferred embodiments of the present invention. To assess the full scope of the invention the claims should be looked to as these preferred embodiments are not intended to be the only embodiments within the scope of the claims.

### DESCRIPTION OF THE DRAWINGS

FIG. 1 illustrates expression levels of a-crystallin containing proteins that are present in Dictyostelium discoideum during its developmental process.

FIG. 2 illustrates a GFP tagged Hsp48 (^{GFP}Hsp48) expressed in Dictyostelium cells.

FIG. 3A illustrates a fluorescent recovery after photobleaching (FRAP) experiment on cells expressing ^{GFP}Hsp48, where the cells contain a phase separated biomolecular condensate in a liquid-like state.

FIG. 3B illustrates a fluorescent recovery after photobleaching (FRAP) experiment on cells expressing ^{GFP}48, where the cells contain a phase separated biomolecular condensate in a solid-like state.

FIG. 4 illustrates the accumulation of polyphosphate on a western blot gel at 0, 6, 12, 16, 18, and 24 hours after induced Dictyostelium development by starvation.

FIG. 5 is an illustration of a biological mesh according to one embodiment of the present invention.

FIG. 6 is a schematic illustration of a phase separated compartment within the biological mesh of FIG. 5, according to one embodiment of the present invention.

FIG. 7 is a sequence listing of a heat shock protein in Dictostelium discodieum.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As used herein, the term “Hsp48” refers to a Dictyostelium dicoideum heat shock protein having an approximate molecular weight expressed in kDa.

As used herein, the term “phase-separation” may be defined as a liquid demixing of Hsp48 and polyphosphate where the Hsp48 and polyphosphate form a membrane-less compartment.

For the purposes of illustration, it can be viewed similar to a drop of oil being added to water. The oil will separate to form a droplet and not mix with the water. Unlike oil in water, the phase-separation used herein may exist in a gradient of fluid-like states. For example, it may range from a fluid-like state to a glass-like state.

### Mechanism of Proteostasis

The present disclosure relates to our investigations of the mechanisms and molecular compositions that allow certain amoeba, such as Dictyostelium, to maintain proteostasis during the developmental process. As will be detailed below, deciphering these mechanisms and molecular compositions has led Applicants to the discovery of a new biomaterial that may be used in a myriad of industrial and pharmaceutical applications.

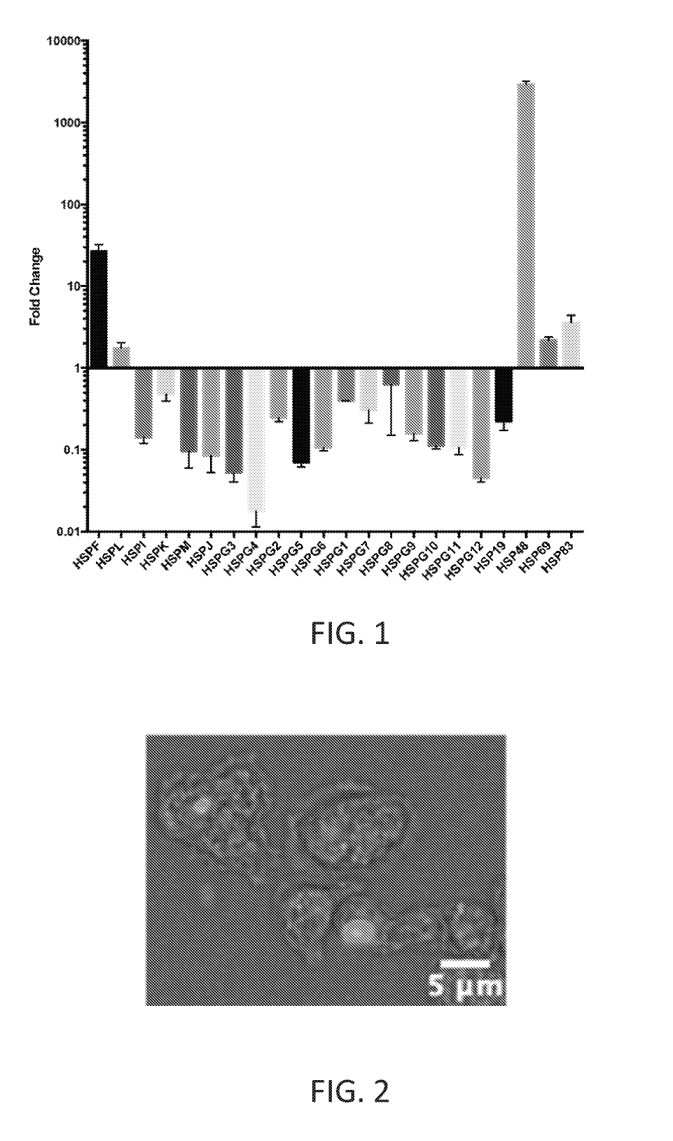

To investigate which protein quality control pathways play a protective role during Dictyostelium development, expression levels of a-crystallin containing proteins were measured. FIG. 1 shows that one previously uncharacterized a-crystallin containing protein, Hsp48, is identified whose transcript levels are induced nearly 10,000-fold in fruiting bodies compared to the amoeba. Unlike most other a-crystallin containing proteins, such as HSPG1 and HSPG2, Hsp48 expression did not increase upon heat stress, suggesting a more selective role in development.

To investigate the role of Hsp48 in the developmental process, Hsp48 knockout cells were generated that had induced development by starvation. Consistent with Hsp48 playing an important role during Dictyostelium development, Hsp48 knockout cells failed to develop. Together, these data demonstrate an essential role for HSP48 during development, but not during vegetative growth.

Without limiting the present disclosure to a particular mechanism, it is reasoned that because Hsp48 is an a-crystallin domain containing protein, it helps maintain proteostasis during Dictyostelium development. One common indication of a failure of the proteostatic network to suppress protein aggregation is the accumulation of ubiquitin positive puncta. To determine if deletion of Hsp48 would led to a failure of proteostasis during Dictyostelium development, ubiquitin immunostaining was performed on wild-type and Hsp48−/− vegetative and developed cells. Because HSP48 −/− cells failed to completely develop, HSP48 −/− cells were allowed to reach their most developed state and wild type cells at the corresponding developmental time point. While no ubiquitin positive puncta could be identified in wild-type cells, Hsp48−/− cells formed ubiquitin-positive puncta during development.

To confirm that the ubiquitin positive puncta observed in Hsp48−/− cells were formed by protein aggregation, differential centrifugation experiments were then performed. The soluble and insoluble fractions were probed for the presence of ubiqutianted proteins. Consistent with the ubiquitin puncta observed in protein aggregates, a large increase in polyubiquitinated species in the insoluble fraction was observed. Together, these data are consistent with an important role for Hsp48 in maintaining proteostasis during Dictyostelium development.

Different protein quality control pathways exist in various cellular compartments including the endoplasmic reticulum, the nucleus, the cytoplasm, and the spore coat in developing bacteria. To determine Hsp48's cellular localization, a GFP tagged Hsp48 (^{GFP}Hsp48) was generated and expressed in Dictyostelium cells. ^{GFP}Hsp48 was present in the cytosol, however it was not diffuse. As shown in FIG. 2, the ^{GFP}Hsp48 formed puncta in the cells.

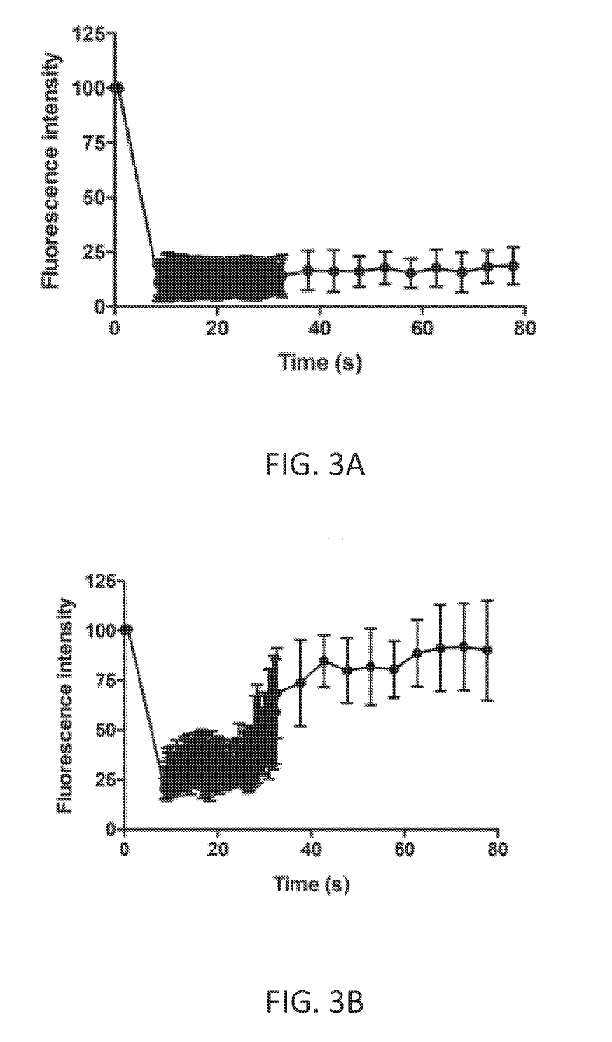

The ^{GFP}Hsp48 puncta did not appear to co-localize to any organelle, but they did appear to form spherical structures. In addition to protein aggregates which often form spherical puncta, biomolecular condensates have recently been shown to form liquid-liquid phase separated spheres (7). Biomolecular condensates may be present in a gradient of states ranging from highly dynamic liquid like states, to more glass like solid states. To discriminate between the two states, fluorescent recovery after photobleaching (FRAP) experiments were performed on cells expressing ^{GFP}Hsp48. Multiple states for ^{GFP}Hsp48 were observed with one population behaving more liquid like, as shown in FIG. 3A, while a second population behaved more solid like, as shown in FIG. 3B. Together these data suggest that Hsp48 forms a biomolecular condensate in the cytoplasm of Dictyostelium.

One common characteristic of proteins that form biomolecular condensates is the presence of an intrinsically disordered domain. To determine if Hsp48 has an intrinsically disordered domain, in silico analysis of Hsp48's sequence was performed using multiple algorithms, including PONDR, FoldIndex, and IUPred. In all cases the C-terminus of Hsp48 was predicted to be intrinsically disordered. To determine if Hsp48's intrinsically disordered domain was responsible for driving phase separation, an N-terminal α-crystallin domain (GFPHsp48ΔCterm) and a C-terminal intrinsically disordered domain (GFPHsp48ΔαCrys) were expressed within Dictyostelium to test for the formation of the biomolecular condensate. Similar to wild-type Hsp48, the GFPHsp48ΔαCrys protein formed the biomolecular condensate, while the GFPHsp48ΔCterm construct did not form the biomolecular condensate.

Unlike many organelles and vesicles, the biomolecular condensates form compartments that do not require a lipid bilayer. To determine if Hsp48 puncta required a lipid bilayer we next disrupted lipid bilayers with detergent and assessed the ability of cells expressing either GFPHSP48, GFPHSP48ΔαCrys, or GFPHsp48ΔCterm to form GFP puncta. Consistent with our in vivo data, GFPHsp48 and GFPHSP48ΔαCrys puncta remained after cell lysis, while GFPHsp48ΔCterm was diffuse. Together, these data demonstrates that Hsp48 forms an biomolecular condensate driven by its C-terminus.

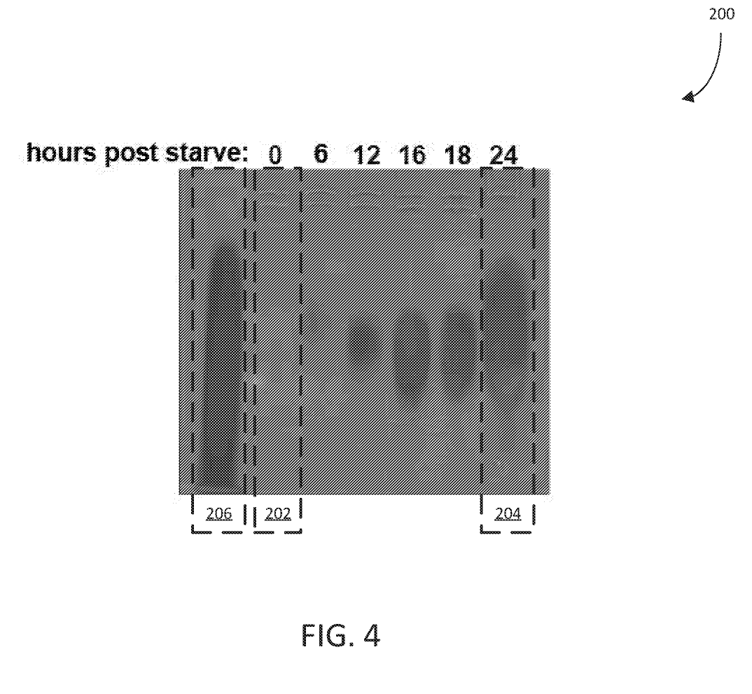

Furthermore, it is found that the formation of the biomolecular condensate is driven by an upregulation of polyphosphate during Dictyostelium development. FIG. 4 shows the accumulation of polyphosphate 200 on a western blot gel at 0, 6, 12, 16, 18, and 24 hours after induced Dictyostelium development by starvation. The accumulation of polyphosphate 200 is tracked from a vegetative state 202 to a fruiting body 204 and is compared to a polymeric reference material 206. In the illustrated embodiment, the polymeric reference material 206 consists of polyphosphate having polyphosphate monomer units [PO₃⁻]ₙ, where n is approximately 100. These data suggest that the accumulation of polyphosphate 200 increases during Dictyostelium development from the vegetative state 202 to the fruiting body 204, thereby suggesting polyphosphate plays a role in the formation of the biomolecular condensate. Furthermore, it is found that removal of polyphosphate kinase 1, the enzyme responsible for polyphosphate production, prevents the formation of the biomolecular condensate in the fruiting body 204.

The natural biomolecular condensate that forms during Dictyostelium development forms phase-separated compartments having a mean diameter between approximately 10 to 100 nm. Given that the phase-separated compartments of the biomolecular condensate occur in the cytoplasm, the biomolecular condensate may include impurities entrained within the biomolecular condensate. Some of these impurities may include naturally occurring proteins and ions dissolved in the cytosol, organelles, and cytoplasmic inclusions (glycogen, lipids, pigments, crystals, etc). The biomolecular condensate also contains a number of heat shock proteins that stabilize misfolded proteins, and act as holdases that prevent further protein aggregation.

### The Present Disclosure

In one general embodiment, the present disclosure relates to using the knowledge gained from the formation of biomolecular condensates during Dictyostelium development to enhance liquid-liquid phase separation in samples for the formation of improved biomaterials, bioelectronics, and drug delivery platforms. As will be detailed below, samples with enhanced liquid-liquid phase separation are herein referred to as a biological mesh.

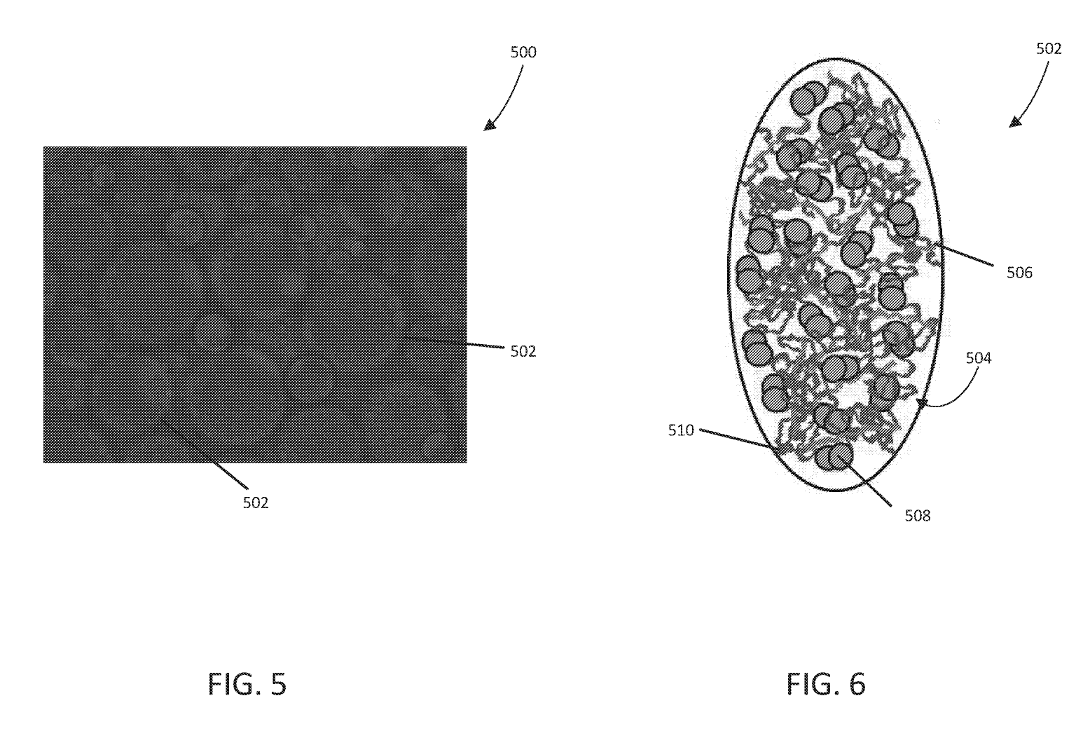

FIG. 5 shows a biological mesh 500 according to one embodiment of the present disclosure. The biological mesh 500 comprises one or more phase-separated compartments 502 configured within the sample. The phase separated compartments 502 comprise non-membrane-bound compartments that are present in a gradient of states ranging from highly dynamic liquid-like states to glass-like solid states. In some aspects, the sample includes a solvent or a solution that comprises a liquid phase. In one non-limiting example, the solution may comprise a denaturing buffer. The phase-separated compartments 502 may have a mean diameter that is less than 10 nm. In some embodiments, the mean diameter ranges between 1 to 9 nm, or between 1 to 8 nm, or between 1 to 7 nm, or between 1 to 6 nm, or between 1 to 5 nm. The phase-separated compartments 502 may have a mean diameter that is greater than 200 nm, or is greater than 250 nm, or is great than 300 nm. In other embodiments, the phase-separated compartments 502 have a mean diameter that ranges between 200 nm and 5 μm.

FIG. 6 shows a schematic illustration of a phase separated nanoparticle 502 according to one embodiment of the present disclosure. The phase separated nanoparticle 502 may comprise one or more a Dictyostelium discoideum Hsp48 protein 504 and a polymeric material 506 having a negative charge. In some aspects, the Dictyostelium discoideum Hsp48 protein 504 comprises a C-terminus domain 508 and an intrinsically disordered domain 510. While, in other aspects, the phase separated nanoparticle 502 comprises at least a portion of the C-terminus domain 508 and the polymeric material 506 having a negative charge. In one non-limiting example, the C-terminus domain 508 comprises residues 138-416 of the Dictyostelium discoideum Hsp48 protein 504. The state of the phase separated compartments 502 may be controlled from a liquid-like state to a glass-like solid state by increasing or decreasing the concentrations of the polymeric material 506 and at least a portion of the C-terminus domain 508.

In one non-limiting example, the polymeric material 506 may comprise polyphosphate having a number of monomer units [PO₃⁻]ₙ. In some aspects, the number of monomer units is greater than 25, is greater than 50, or is greater than 75, or is greater than 100, or is greater than 150, or is greater than 200, or is greater than 300. In other aspects, the number of monomer units ranges from 200-2000. In some aspects, the number of monomer units ranges from 300 to 1900, or ranges from 400 to 1800, or ranges from 500 to 1700, or ranges from 600 to 1600, or ranges from 700 to 1500. The concentration of the polymeric material 506 in the biological mesh 500 may range from 100 μM to 5 mM.

Other suitable polymeric materials 506 may comprise polyelectrolytes or ionomers. The polyelectrolyte or ionomer may comprise a repeating unit coupled to one or more electrolyte group. In some aspects, the electrolyte group comprises a negative charge. In other aspects, the electrolyte group may include functional groups such as —COOH, —SO₃H, —PO₃H₂, and mixtures thereof. In some aspects, the repeating unit is derived from ethylene, propylene, butylene, pentylene, and the like.

In some non-limiting examples, the polymeric material 506 comprises polyacrylic acid, polystyrene sulfonate, and mixtures thereof. In other non-limiting examples, the polymeric material 506 comprises pectin, alginate, carboxymethyl cellulose, polypeptides, and mixtures thereof.

In some aspects, a concentration of the polymeric material 506 having a negative charge may be controlled within the biological mesh 500 such that the polymeric material 506 has a western blot densitometry that is greater than a reference western blot densitometry, where the reference western blot densitometry includes a reference polymeric material taken from a phase separated sample of a Dictyostelium discoideum amoeba.

It is contemplated that other Dictyostelium discoideum heat shock proteins may also be suitable for the biological mesh 500. Suitable Dictyostelium discoideum heat shock proteins may range from Hsp45 to Hsp 55.

The biological mesh 500 may be used in a myriad of applications. In one embodiment, the biological mesh may be used as a drug, vaccine, and gene delivery platform. In this instance, the biological mesh 500 may further comprise a therapeutic substance. In some aspects, the therapeutic substance may be chemically coupled to the one or more Dictyostelium discoideum Hsp48 protein 504. For example, the therapeutic substance may be chemically coupled to the intrinsically disordered domain 510 or the C-terminus domain 508 of the Dictyostelium discoideum Hsp48 protein 504. In other aspects, the therapeutic substance is chemically coupled to the polymeric material 506.

Proteins may be expressed as fusions to the C-terminus domain 508 of the Dictyostelium discoideum Hsp48 protein 504. In some aspects, the therapeutic substance is be entrained within the biological mesh 500. The biological mesh 500 may be used to deliver the therapeutic substance to a region of interest within a subject, such as particular cells or organs. For example, the biological mess 500 may be used to target therapeutics to specific cell types by tagging proteins that bind specific receptors with the C-terminus domain 508 of the Dictyostelium discoideum Hsp48. Suitable therapeutics for the biological mesh 500 may include enzymes, pharmaceutical agents, plasmids, polyneucleotides, polypeptides, antibodies, and the like. In one illustrative example, an antibody can be coupled to the C-terminus domain 508 of the Dictyostelium discoideum Hsp48 protein, and chemotherapies may be coupled to the polymeric material 506 within the biological mesh 500. The biological mesh 500 can then be used to target specific cancer cells within a subject, such as a human or an animal.

In a similar manner described above, the biological mesh 500 may also be used as a delivery platform to a region of interest in applications nutritional supplements, radiation emitting products, imaging and contrast agents, wound healing agents, restorative dentistry, and cosmetics.

The biological mesh 500 may also be used in materials science applications such as in the formation of biomaterials and bioelectronics. In some aspects, several biological meshes 500 may be coupled together to form nano- or micro-structures. Similarly, several biological meshes 500 may be coupled together to form molecularly imprinted polymers. The biological mesh 500 may also be used in the production of resins. The biological mesh 500 may also be doped with a conductive material, such as a metal, to produce a biologically conductive material. It is contemplated that the biologically conductive material may be used to create resistors, biosensors, semiconductors, and surface coatings.

Biological materials produced with the biological mesh 500 offer several advantages such as being natural, biodegradable, and non-toxic. The biological mesh 500 is flexible in construction, and may be selectively tuned to have a dynamic liquid-like state or a glass-like solid state. The biological mesh 500 may be used to improve selectivity in delivering therapeutics to particular target regions, such as tissues, cells, and receptors.

In one embodiment, the present disclosure relates to a method of manufacturing a biological mesh 500. The biological mesh 500 may be produced by adding a Dictyostelium discoideum Hsp48 protein 504 and a polymeric material 506 to a solution having a liquid phase. The Dictyostelium discoideum Hsp48 protein 504 and the polymeric material 506 are added to the solution until at least a portion of the solution undergoes a phase separation to generate the biological mesh 500. In some aspects, a fixed amount of the Dictyostelium discoideum Hsp48 protein 504 is first added to the solution and subsequently the polymeric material 506 is added until the biological mesh 500 is produced. In other aspects, a fixed amount of the polymeric material 506 is added to the solution, and Hsp48 protein is added to the solution until the biological mesh 500 is produced.

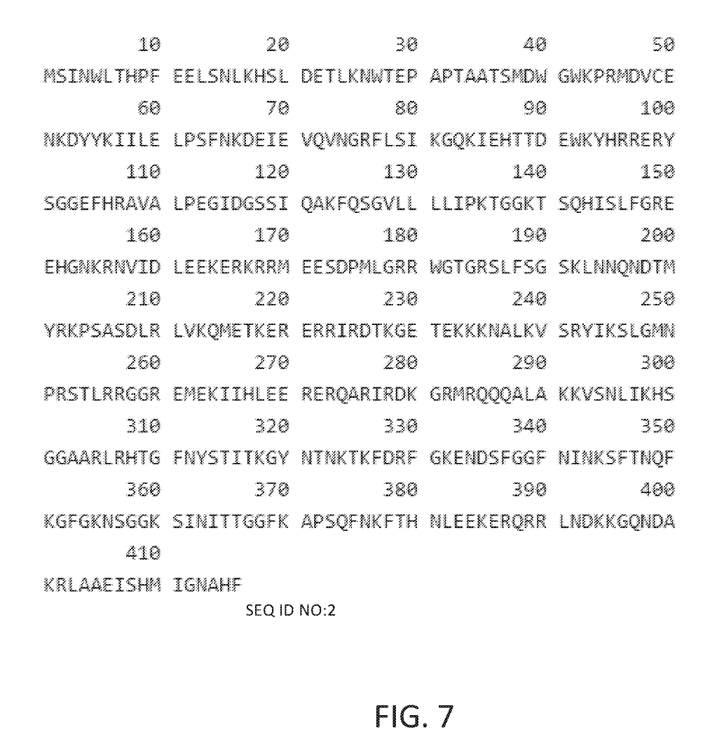

The Dictyostelium discoideum Hsp48 protein 504 may be produced through protein expression in an organism. The full sequence of the Dictyostelium discoideum Hsp48 protein 504 is shown in FIG. 7. One may wish to use only a portion of the Hsp48 gene. In one aspect, only residues 138-416 of the Hsp48 protein are used. A sequence for residues 138-416 is shown in Table 1. It is contemplated that smaller fragments of the Hsp48 protein may also work in accordance with the various aspects of the present disclosure.

TABLE 1
Exemplary fragments of the Hsp48 gene.

| Sequence | |
|---|---|
| ID | Sequence listing |
| SEQ ID: | KTSQHISLFGREEHGNKRNVIDLEEKERKRRMEESDPMLG |
| NO 1 | WRRGTGRSLFSGSKLNNQNDTMYRKPSASDLRLVKQMETK |
| | ERERRIRDTKGETEKKKNALKVSRYIKSLGMNPRSTLRRG |
| | GREMEKIIHLEERERQARIRDKGRMRQQQALAKKVSNLIK |
| | HSGGAARLRHTGFNYSTITKGYNTNKTKEDREGKENDSEG |
| | GENINKSFTNQFKGFGKNSGGKSINTTTGGFKAPSQFNKF |
| | THNLEEKERQRRLNDKKGQNDAKRLAAEISHMIGNAHF |

In some aspects, E. coli cells containing a plasmid that encodes Hsp48, or portions of HsP48, are grown in a suitable broth placed in a shaking incubator. A suitable broth may include Lauria broth at a temperature between 20 degrees and 40 degrees Celsius. The E. coli cells are then shaken in the shaking incubator until it reaches an optical density of approximately 0.6.

Isopropyl β-D-1-thiogalactopyranoside may then be added and the temperature may be reduced. In some aspects, the temperature is reduced to approximately 16 degree Celcius for 18 hours. Cells may then be pelleted and lysed in cold denaturing buffer. In one non-limiting example, the denaturing buffer comprises 8M Urea, 500 mM NaCl, and 20 mM Soduium Phosphate pH 7.2. The cell solution is then sonicated and spun down. In some aspects, the cell solution is sonicated at least three times for approximately 30 seconds. In some aspects, the cell solution is spun down at 20,000×G for approximately 30 minutes in a JA-20 rotor. The JA-20 rotor may be precooled to approximately 4° C.

The lysate may be incubated with Ni—NTA beads for anywhere between 3 and 18 hours. In some aspects, the beads are washed multiple times with denaturing buffer. Hsp48 can then be eluated with elution buffer. In one non-limiting example, the elution buffer comprises 8M Urea, 50 mM NaCl, 20 mM Sodium Phosphate (at ˜pH 7.2) and 300 mM Immidazole (at ˜pH 8.0). In some aspects, elutions are performed with 1 mL of buffer for every 1 mL of Ni—NTA beads used. Eluted protein may then be concentrated. In some aspects, the protein is concentrated to a concentration of greater than 10 mg/mL. In some aspects, the Hsp48 protein is combined with denaturing buffer and polyphosphate to produce the biological mesh.

In another embodiment, Hsp48 containing plasmids may be electroporated into Dictyostelium and expressed. In some aspects, Hsp48 protein is expressed with passaging to maintain cell numbers between approximately 1×10⁴ and 1×10⁷ cells per milliliter. Purification protocols similar to those described above may be used to isolate Hsp48.

Using the methods described above, one non-limiting composition of the biological mesh 500 may include 80 microliters of a 40 mg/mL Hsp48 solution with 20 microliters of 10 millimolar polyphosphate in 50 mM Tris pH 7.5, 50 mM NaCl.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

### REFERENCES

1. Cotter D A, Raper K B. Properties of Germinating Spores of Dictyostelium discoideum. Journal of Bacteriology. 1968; 96(5):1680-9. PubMed PMID: PMC315227.
2. Santarriaga S, Petersen A, Ndukwe K, Brandt A, Gerges N, Bruns Scaglione J, Scaglione K M. The Social Amoeba Dictyostelium discoideum Is Highly Resistant to Polyglutamine Aggregation. Journal of Biological Chemistry. 2015; 290(42):25571-8. doi: 10.1074/jbc.M115.676247.
3. Malinovska L, Palm S, Gibson K, Verbavatz J-M, Alberti S. Dictyostelium discoideum has a highly Q/N-rich proteome and shows an unusual resilience to protein aggregation. Proceedings of the National Academy of Sciences of the United States of America. 2015; 112(20):E2620-E9. doi: 10.1073/pnas.1504459112. PubMed PMID: PMC4443358.
4. Livermore T M, Chubb J R, Saiardi A. Developmental accumulation of inorganic polyphosphate affects germination and energetic metabolism in Dictyostelium discoideum. Proceedings of the National Academy of Sciences of the United States of America. 2016; 113(4):996-1001. doi: 10.1073/pnas.1519440113. PubMed PMID: PMC4743807.
5. Zhang H, Gomez-Garcia M R, Brown M R W, Kornberg A. Inorganic polyphosphate in Dictyostelium discoideum: Influence on development, sporulation, and predation. Proceedings of the National Academy of Sciences of the United States of America. 2005; 102(8):2731-5. doi: 10.1073/pnas.0500023102. PubMed PMID: PMC549442.
6. Riback J A, Katanski C D, Kear-Scott J L, Pilipenko E V, Rojek A E, Sosnick T R, Drummond D A. Stress-Triggered Phase Separation Is an Adaptive, Evolutionarily Tuned Response. Cell. 168(6):1028-40.e19. doi: 10.1016/j.cell.2017.02.027.
7. Hyman A A, Weber C A, Juelicher F. Liquid-Liquid Phase Separation in Biology. In: Schekman R, Lehmann R, editors. Annual Review of Cell and Developmental Biology, Vol 30. Palo Alto: Annual Reviews; 2014. p. 39-58.
8. Kato M, Han T W, Xie S, Shi K, Du X, Wu L C, Mirzaei H, Goldsmith E J, Longgood J, Pei J, Grishin N V, Frantz D E, Schneider J W, Chen S, Li L, Sawaya M R, Eisenberg D, Tycko R, McKnight S L. Cell-free formation of RNA granules: Low complexity sequence domains form dynamic fibers within hydrogels. Cell. 2012; 149(4):753-67. doi: 10.1016/j.cell.2012.04.017.
9. Patel A, Lee Hyun O, Jawerth L, Maharana S, Jahnel M, Hein Marco Y, Stoynov S, Mahamid J, Saha S, Franzmann Titus M, Pozniakovski A, Poser I, Maghelli N, Royer Loic A, Weigert M, Myers Eugene W, Grill S, Drechsel D, Hyman Anthony A, Alberti S. A Liquid-to-Solid Phase Transition of the ALS Protein FUS Accelerated by Disease Mutation. Cell.162(5):1066-77. doi: 10.1016/j.cell.2015.07.047.
10. Lin Y, Protter David S W, Rosen Michael K, Parker R. Formation and Maturation of Phase-Separated Liquid Droplets by RNA-Binding Proteins. Molecular Cell.60(2):208-19. doi: 10.1016/j.molcel.2015.08.018.
11. Guo L, Shorter J. It&#x2019; s Raining Liquids: RNA Tunes Viscoelasticity and Dynamics of Membraneless Organelles. Molecular Cell.60(2):189-92. doi: 10.1016/j.molcel.2015.10.006.
12. Bergeron-Sandoval L-P, Safaee N, Michnick Stephen W. Mechanisms and Consequences of Macromolecular Phase Separation. Cell.165(5):1067-79. doi: 10.1016/j.cell.2016.05.026.
13. Aguzzi A, Altmeyer M. Phase Separation: Linking Cellular Compartmentalization to Disease. Trends in Cell Biology. 2016; 26(7):547-58. doi: http://dx.doi.org/10.1016/j.tcb.2016.03.004.
14. Xiang S, Kato M, Wu Leeju C, Lin Y, Ding M, Zhang Y, Yu Y, McKnight Steven L. The LC Domain of hnRNPA2 Adopts Similar Conformations in Hydrogel Polymers, Liquid-like Droplets, and Nuclei. Cell.163(4):829-39. doi: 10.1016/j.cell.2015.10.040.
15. Murakami T, Qamar S, Lin Julie Q, Schierle Gabriele S K, Rees E, Miyashita A, Costa Ana R, Dodd Roger B, Chan Fiona T S, Michel Claire H, Kronenberg-Versteeg D, Li Y, Yang S-P, Wakutani Y, Meadows W, Ferry Rodylyn R, Dong L, Tartaglia Gian G, Favrin G, Lin W-L, Dickson Dennis W, Zhen M, Ron D, Schmitt-Ulms G, Fraser Paul E, Shneider Neil A, Holt C, Vendruscolo M, Kaminski Clemens F, St George-Hyslop P. ALS/FTD Mutation-Induced Phase Transition of FUS Liquid Droplets and Reversible Hydrogels into Irreversible Hydrogels Impairs RNP Granule Function. Neuron. 2015; 88(4):678-90. doi: http://dx.doi/10.1016/j.neuron.2015.10.030.
16. Molliex A, Temirov J, Lee J, Coughlin M, Kanagaraj Anderson P, Kim Hong J, Mittag T, Taylor J P. Phase Separation by Low Complexity Domains Promotes Stress Granule Assembly and Drives Pathological Fibrillization. Cell. 2015; 163(1):123-33. doi: http://dx.doi.org/10.1016/j.cell.2015.09.015.
17. Nott Timothy J, Petsalaki E, Farber P, Jervis D, Fussner E, Plochowietz A, Craggs T D, Bazett-Jones David P, Pawson T, Forman-Kay Julie D, Baldwin Andrew J. Phase Transition of a Disordered Nuage Protein Generates Environmentally Responsive Membraneless Organelles. Molecular Cell. 2015; 57(5):936-47. doi: http://dx.doi.org/10.1016/j.mol.cel.2015.01.013.
18. Pak Chi W, Kosno M, Holehouse Alex S, Padrick Shae B, Mittal A, Ali R, Yunus Ali A, Liu David R, Pappu Rohit V, Rosen Michael K. Sequence Determinants of Intracellular Phase Separation by Complex Coacervation of a Disordered Protein. Molecular Cell. 2016; 63(1):72-85. doi: http://dx.doi.org/10.1016/j.molcel.2016.05.042.

19. Altmeyer M, Neelsen K J, Teloni F, Pozdnyakova I, Pellegrino S, Grøfte M, Rask M-B D, Streicher W, Jungmichel S, Nielsen M L, Lukas J. Liquid demixing of intrinsically disordered proteins is seeded by poly(ADP-ribose). Nature Communications. 2015; 6:8088. doi: 10.1038/ncomms9088. PubMed PMID: PMC4560800.
20. Jain S, Wheeler Joshua R, Walters Robert W, Agrawal A, Barsic A, Parker R. ATPase-Modulated Stress Granules Contain a Diverse Proteome and Substructure. Ce11.164(3):487-98. doi: 10.1016/j.cell.2015.12.038.
21. Wheeler J R, Matheny T, Jain S, Abrisch R, Parker R. Distinct stages in stress granule assembly and disassembly. eLife. 2016; 5:e18413. doi: 10.7554/eLife.18413. PubMed PMID: PMC5014549.
22. Moerman A M, Klein C. Developmental Regulation of Hsp32, a Small Heat Shock Protein inDictyostelium discoideum. Experimental Cell Research. 1997; 237(1):149-57. doi: http://dx.doi.org/10.1016/excr.1997.3774.
23. Sawarkar R, Roy N, Rao S, Raman S, Venketesh S, Suguna K, Tatu U. Heat Shock Protein 90 Regulates Development in Dictyostelium discoideum. Journal of Molecular Biology. 2008; 383(1):24-35. doi: https://doi.org/10.1016/j.jmb.2008.08.006.
24. Otto GP, Wu M Y, Kazgan N, Anderson O R, Kessin R H. Dictyostelium macroautophagy mutants vary in the severity of their developmental defects. The Journal of biological chemistry. 2004; 279(15):15621-9. Epub 2004/01/23. doi: 10.1074/jbc.M311139200. PubMed PMID: 14736886.
25. Otto G P, Wu M Y, Kazgan N, Anderson O R, Kessin R H. Macroautophagy is required for multicellular development of the social amoeba Dictyostelium discoideum. The Journal of biological chemistry. 2003; 278(20):17636-45. Epub 2003/03/11. doi: 10.1074/jbc.M212467200. PubMed PMID: 12626495.
26. Lindsey D F, Amerik A, Deery W J, Bishop J D, Hochstrasser M, Gomer R H. A deubiquitinating enzyme that disassembles free polyubiquitin chains is required for development but not growth in Dictyostelium. The Journal of biological chemistry. 1998; 273(44):29178-87. Epub 1998/10/24. PubMed PMID: 9786928.
27. Gray M J, Wholey W Y, Wagner N O, Cremers C M, Mueller-Schickert A, Hock N T, Krieger A G, Smith E M, Bender R A, Bardwell J C, Jakob U. Polyphosphate is a primordial chaperone. Mol Cell. 2014; 53(5):689-99. Epub 2014/02/25. doi: 10.1016/j.molcel.2014.01.012. PubMed PMID: 24560923; PMCID: PMC3996911.
28. Cremers Claudia M, Knoefler D, Gates S, Martin N, Dahl J-U, Lempart J, Xie L, Chapman Matthew R, Galvan V, Southworth Daniel R, Jakob U. Polyphosphate: A Conserved Modifier of Amyloidogenic Processes. Molecular Cell.63(5):768-80. doi: 10.1016/j.molcel.2016.07.016.
29. Munder M C, Midtvedt D, Franzmann T, Nüske E, Otto O, Herbig M, Ulbricht E, Muller P, Taubenberger A, Maharana S, Malinovska L, Richter D, Guck J, Zaburdaev V, Alberti S. A pH-driven transition of the cytoplasm from a fluid- to a solid-like state promotes entry into dormancy. eLife. 2016; 5:e09347. doi: 10.7554/eLife.09347. PubMed PMID: PMC4850707.
30. Joyner R P, Tang J H, Helenius J, Dultz E, Brune C, Holt L I, Huet S, Muller D J, Weis K. A glucose-starvation response regulates the diffusion of macromolecules. Elife. 2016; 5. Epub 2016/03/24. doi: 10.7554/eLife.09376. PubMed PMID: 27003290; PMCID: PMC4811765.
31. Parry Bradley R, Surovtsev Ivan V, Cabeen Matthew T, O'Hern Corey S, Dufresne Eric R, Jacobs-Wagner C. The Bacterial Cytoplasm Has Glass-like Properties and Is Fluidized by Metabolic Activity. Cell.156(1):183-94. doi: 10.1016/j.cell.2013.11.028.

We claim:

1. A biological mesh, the biological mesh comprising:
one or more heat shock protein domains, wherein the one or more heat shock protein domains comprise at least a portion of a C-terminus of a Dictyostelium discoideum Hsp48 protein; and
a polymeric material having a negative charge, the polymeric material comprising polyphosphate having a number of monomer units [PO₃⁻]ₙ, wherein n is greater than 100.

2. The biological mesh of claim 1, wherein the polymeric material polyphosphate having a number of monomer units [PO₃⁻]ₙ, wherein n ranges from 200-2000.

3. The biological mesh of claim 1, wherein the heat shock protein domain further comprises an alpha-crystalline domain and an intrinsically disordered region.

4. The biological mesh of claim 1, wherein the heat shock protein comprises a Dictyostelium discoideum Hsp48 protein.

5. The biological mesh of claim 1, wherein the C-terminus domain comprises residues 138-416 of the Dictyostelium discoideum Hsp48 protein.

6. The biological mesh of claim 1, wherein the biological mesh further comprises a therapeutic substance.

7. The biological mesh of claim 1, wherein the therapeutic substance is coupled to the one or more heat shock protein domains.

8. The biological mesh of claim 1, wherein the therapeutic substance is coupled to the polymeric material.

9. The biological mesh of claim 1, wherein the therapeutic substance is entrained within the biological mesh.

10. The biological mesh of claim 6, wherein the therapeutic substance is selected from the group consisting of an enzyme, a pharmaceutical agent, a plasmid, a polyneucleotide, a polypeptide, and an antibody.

11. The biological mesh of claim 1, wherein more than one of the biological mesh are coupled to form nano- or micro-structures.

12. The biological mesh of claim 1, wherein more than one of the biological mesh are coupled to form molecularly imprinted polymers.

13. The biological mesh of claim 1, wherein the biological mesh is doped with a conductive material to produce a biologically conductive material.

14. The biological mesh of claim 13, wherein the conductive material includes a metal.

15. A biological mesh, the biological mesh comprising:
one or more heat shock protein domains, wherein the one or more heat shock protein domains comprise at least a portion of a C-terminus of a Dictyostelium discoideum Hsp48 protein; and
a polymeric material comprising a polyelectrolyte or an ionomer having a repeating unit coupled to one or more electrolyte group, wherein the electrolyte group is selected from the group consisting of —COOH, —SO₃H, and —PO₃H₂.

16. The biological mesh of claim 10, wherein the polymeric material is selected from polyacrylic acid, polystyrene sulfonate, and mixtures thereof.

17. The biological mesh of claim 10, wherein the polymeric material comprises pectin, alginate, carboxymethyl cellulose, polypeptides, and mixtures thereof

18. A biological mesh, the biological mesh comprising:
one or more heat shock protein domains, wherein the one or more heat shock protein domains comprise at least a portion of a C-terminus of a Dictyostelium discoideum Hsp48 protein; and
a polymeric material having a negative charge, the polymeric material having a western blot densitometry that is greater than a reference western blot densitometry, wherein the reference western blot densitometry includes a phase separated sample taken from a Dictyostelium discoideum amoeba.

19. A method for forming a biological mesh, the method comprising:
(a) providing a solution, the solution having a liquid phase; and
(b) adding at least a portion of a C-terminus domain of a Dictyostelium discoideum Hsp48 protein and a polymeric material having a negative charge to the solution, the solution undergoing a phase separation to generate the biological mesh.


SEQUENCE LISTING (2 sequences)

SEQ ID NO: 1 - protein, 278 aa, Dictyostelium discoideum
KTSQHISLFGREEHGNKRNVIDLEEKERKRRMEESDPMLGRRWGTGRSLFSGSKLNNQNDTMYRKPSASDLRLVKQMETKERERRIRDTKGETEKKKNALKVSRYIKSLGMNPRSTLRRGGREMEKIIHLEERERQARIRDKGRMRQQQALAKKVSNLIKHSGGAARLRHTGFNYSTITKGYNTNKTKFDRFGKENDSFGGFNINKSFTNQFKGFGKNSGGKSINITTGGFKAPSQFNKFTHNLEEKERQRRLNDKKGQNDAKRLAAEISHMIGNAHF

SEQ ID NO: 2 - protein, 416 aa, Dictyostelium discoideum
MSINWLTHPFEELSNLKHSLDETLKNWTEPAPTAATSMDWGWKPRMDVCENKDYYKIILELPSFNKDEIEVQVNGRFLSIKGQKIEHTTDEWKYHRRERYSGGEFHRAVALPEGIDGSSIQAKFQSGVLLLLIPKTGGKTSQHISLFGREEHGNKRNVIDLEEKERKRRMEESDPMLGRRWGTGRSLFSGSKLNNQNDTMYRKPSASDLRLVKQMETKERERRIRDTKGETEKKKNALKVSRYIKSLGMNPRSTLRRGGREMEKIIHLEERERQARIRDKGRMRQQQALAKKVSNLIKHSGGAARLRHTGFNYSTITKGYNTNKTKFDRFGKENDSFGGFNINKSFTNQFKGFGKNSGGKSINITTGGFKAPSQFNKFTHNLEEKERQRRLNDKKGQNDAKRLAAEISHMIGNAHF